(19) United States
(12) Patent Application Publication
(10) Pub. No.: US 2008/0305494 A1
(43) Pub. Date: Dec. 11, 2008

(54) Chemokine Ccl18 as a Biomarker

(76) Inventors: Jose M. Carballido Herrera, Perchtoldsdorf (AT); Claudia Guenther, Dresden (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/592,831

(22) PCT Filed: Mar. 21, 2005
(86) PCT No.: PCT/EP2005/002986

§ 371 Date: Aug. 26, 2008

Related U.S. Application Data
(60) Provisional application No. 60/555,110, filed on Mar. 22, 2004.

(51) Int. Cl. G01N 33/53

(52) U.S. Cl. 435/7.1

(57) ABSTRACT

The invention relates to a diagnostic tool, e.g. to the chemokine CCL18 as a biomarker in a sample of a body fluid of an individual and its use in various methods.

The present invention relates to a diagnostic tool, e.g. to the chemokine CCL18 for use as a biomarker and its use in various methods.

The regulation of leukocyte trafficking during homeostatic and pathologic responses is achieved in great part by chemokines. Chemokines comprise a large group of homologous proteins that exert their biological effects by interactions with cell surface heptahelical G protein-coupled receptors. CCL18, also designated DC-CK1, PARC, AMAC-1 and MIP-4, is a human chemokine structurally related to CCL3. So far, CCL18 has no mouse equivalent and its receptor is not known. CCL18 is constitutively expressed in germinal centers and tonsils by dendritic cells (DC) and was shown to attract mainly naive T cells, CD38 mantle zone B lymphocytes and DC. The production of CCL18 by antigen presenting cells (APC) is enhanced by Th2 cytokines like IL-4, IL-13 and suppressed by IFN-γ.

Atopic dermatitis (AD) is a T helper 2 (Th2) mediated, chronic inflammatory skin disease characterized by eczematous skin lesions, which are e.g. characterized by intensely pruritic erythematous papules associated with epidermal intercellular edema. Histopathologically, skin lesions of AD reveal a mononuclear cell infiltrate, consisting of macrophages, increased numbers of IgE-bearing Langerhans cells (LC), inflammatory dendritic epidermal cells, eosinophils and CD4⁺ Th cells (see e.g. Leung D Y., J. Allergy Clin. Immunol. 2000, 105:860-76). In acute lesions these Th cells mainly produce Th2 cytokines like IL-4, IL-5 and IL-13, whereas in chronic AD IFN-γ secreting Th cells are also present. The chemokines suggested to mediate the homing of Th cells into AD skin include the CCR4 ligands CCL17, CCL22 and the CCR10 ligand CCL27. However, additional chemotactic signals may be required for the specific recruitment of specialized T cell subpopulations.

Up to now, there are no reliable laboratory markers for AD but AD patients often have elevated IgE levels, allergic reactivity to foods and to other common allergens such as pollens, molds, and Insects. Diagnostic indices, regardless of the type of allergen, useful in comprehending pathological conditions of AD patients and for determining the treatment regimen for the disease are greatly needed in the art. Markers for allergic diseases that are not only less harmful to patients but also capable of readily providing information required for diagnosis would be of great use.

We have found that levels of CCL18 and CCL18-secreting cells, e.g. APC (Including monocytes) and DC, in a body fluid, e.g. in blood, are significantly increased in e.g. AD patients as compared to healthy controls. This and other findings highlight the usefulness of CCL18 as a biomarker.

In one aspect the present invention provides the chemokine CCL18 for use as a biomarker in a sample of a body fluid of an individual.

Biomarker as used in the present invention means that determination (=detection and/or quantification) of CCL18 in a sample of a body fluid of an individual is an indicator for a disorder as such and/or for monitoring the status of a disorder.

The sample of a body fluid of an individual may be derived from blood, e.g. isolated mononuclear cells, or from a blood fraction, e.g. plasma or serum, e.g. serum. In another aspect the present invention provides the use of CCL18 as a biomarker in a blood sample, e.g. serum.

CCL18 as used herein includes full-length CCL18 protein, a CCL18 protein fragment, a mutated CCL18 protein, CCL18 derivatives and CCL18-secreting (producing) cells, e.g. antigen presenting cells (APC) including monocytes and dendritic cells (DC). Fragments, mutants and derivatives of CCL18 are such that the biomarker characteristic of CCL18 is retained.

The use of CCL18 as a biomarker according to the present invention means that CCL18 is determined in said sample of an individual, e.g. with detection means including those as conventional in the field of assays, e.g. immunoassays, such as enzyme linked immunoassays (ELISAs); fluorescence based assays, such as dissociation enhanced lanthanide fluoroimmunoassay (DELFIA) or radiometric assays. Detection means of the present invention include e.g. a molecule which specifically recognizes CCL18, e.g. a molecule which is directly or indirectly detectable. Detection means of the present invention preferably comprise an antibody, Including antibody derivatives or fragments thereof, e.g. an antibody which recognizes CCL18, e.g. a label bearing CCL18 recognizing antibody.

In another aspect the level of CCL18 is determined in using a CCL18 specific antibody.

The label may be one as conventional, e.g. biotin or an enzyme such as alkaline phosphatase (AP), horse radish peroxidase (HRP) or peroxidase (POD) or a fluorescent molecule, e.g. a fluorescent dye, such as e.g. fluorescein isothiocyanate. Preferably the label is biotin. The label bearing molecule, e.g. the label bearing antibody, may be detected according to methods as conventional, e.g. via fluorescence measurement or enzyme detection methods.

In a preferred aspect a CCL18 recognizing antibody is used as a detection mean, e.g. in labeled, such as fluorescent labeled, form.

CCL18-secreting cells in a sample of a body fluid of an individual, e.g. blood, may be determined in using methods as conventional, such as e.g. as described below. Cells may be purified, e.g. separated by a density gradient, from the sample, e.g. blood, and the purified cells obtained are stained. Anti-CCL18 antibodies, e.g. fluorescence labeled anti-CCL18 antibodies, are added to the stained cell preparation, optionally after stimulation of the cells, e.g. with interleukin-4, and the level of CCL18-secreting cells is determined.

Optionally, the CCL18 comprised in the sample or the CCL18 recognizing, e.g. detectable, molecule comprised in the detection means is immobilized on a solid phase. An appropriate solid phase includes e.g. one as conventional, e.g. a plastic plate like a polystyrene or polyvinyl plate, especially a microtiter plate. Also microbeads can be used as a solid phase, e.g. coated microbeads. The solid phase can be coated with a coating material the nature of which depends e.g. on the label comprised in the detection means. The coating material should be able to bind to the label, e.g. the label is biotin and the coating material Includes streptavidin, e.g. covalently bound to the solid phase.

In another aspect the present invention provides the chemokine CCL18 for use as a biomarker in a sample of a body fluid of an Individual for a disorder or disease selected from the group consisting of allergic disease and Th2 mediated disorder.

In another aspect the present invention provides the chemokine CCL18 for use as a biomarker in a sample of a body fluid of an individual for a disorder or disease which is related with atopic dermatitis.

Disorders and diseases as used in the present invention include various kinds and severities of disorders and diseases which are selected from the group consisting of allergic disease and Th2 mediated disorder. Allergic diseases and Th2 mediated disorders include e.g. hypersensitivity pneumonitis, vernal keratoconjunctivitis, contact hypersensitivity and disorders or diseases which are related with atopic dermatitis, including various inflammatory skin diseases, e.g. atopic dermatitis.

In another aspect the present invention provides method for screening and/or in vitro diagnosing of a disorder or disease selected from the group consisting of allergic disease and Th2 mediated disorder in an individual, which method comprises

a) providing a sample of a body fluid of an individual,

b) determining the level of CCL18 in said sample as provided in step a),

c) comparing the level of CCL18 as determined in step b) with a reference level from a sample of a body fluid of a healthy control individual, and

d) screening and/or in vitro diagnosing a disorder or disease selected from the group consisting of allergic disease and Th2 mediated disorder in determining whether the level of said CCL18 as determined in step b) is significantly different from said reference level.

Determination of CCL18 is carried out as described above, e.g. by using a molecule which specifically recognizes the biomarker, e.g. an antibody, an antibody derivative, an antibody fragment, such as e.g. an anti CCL18 antibody, e.g. a commercially available CCL18 specific antibody, e.g. by an immunodiagnostic assay method.

In another aspect the present invention provides a method for monitoring the therapeutic efficacy of the treatment of an individual with a substance which is expected to have an effect on reducing or curing a disorder or disease selected from the group consisting of allergic disease and Th2 mediated disorder, which method comprises determining the level of CCL18 in a sample of a body fluid of said individual and comparing it to the level of CCL18 prior to administration of said substance.

In another aspect the present invention provides a kit for screening and/or in vitro diagnosing of a disorder or disease selected from the group consisting of allergic disease and Th2 mediated disorder in a sample of a body fluid of an individual comprising

a) a molecule which recognizes CCL18 protein or a part thereof, optionally in a labeled form,

b) instructions for use,

c) optionally detection means,

d) optionally a solid phase.

Such kit as provided by the present invention may further comprise a substantial component including an appropriate environment of a sample to be tested and, e.g. appropriate means to determine CCL18 in a sample to be tested.

### DESCRIPTION OF THE FIGURES

FIG. 1: CCL18 expression in AD. Frozen sections of lesional biopsies from AD, psoriasis or healthy skin are stained for CCL18 expression.

FIG. 2: CCL18 expression in AD. Double-immunofluorescence staining with indicated antibodies performed on frozen sections of AD skin biopsies. Original magnification 200× (FIG. 1) and 400× (FIG. 2).

FIG. 3: Serum levels of CCL18. PBMC from AD patients (n=8) and controls (n=13) are stimulated with IL-4. The percentages of CCL18-producing monocytes in PBMC and CCL18-secreting CD11c⁺ blood DC are represented as boxplots. Analysis shows significant differences between monocytes of AD and controls (averages 7.1% and 1.3% respectively, p<0.003) and between DC of AD and controls (averages 5.6% and 1.9% respectively, p<0.001).

FIG. 4: Serum levels of CCL18. CCL18 levels in serum from AD patients (n=36) are significantly higher than those of healthy control (n=28) subjects (averages 34.9 ng/ml and 10.7 ng/ml respectively, p<0.0003).

FIG. 5: Serum levels of CCL18. Graphical distribution of serum levels of CCL18 among AD patients with different severity disease scores (IGA).

FIG. 6: Serum levels of CCL18. Correlation between CCL18 and CCL22 serum levels in AD patients. Filled circles include patients with very mild to severe manifestations (r=0.6, p<0.003). Patients with highest severity scores, represented by empty circles, are excluded from the correlation analysis.

FIG. 7: CCL18 binding of AD derived memory T cells. Immunohistochemistry analysis of CCL18-binding (red) and CD3 expressing (dark blue) cells in AD skin. Counterstained by HE (1000× original magnification).

FIG. 8: CCL18 binding of AD derived memory T cells. CCL18- or CCL22-binding cells using fluorescence (F) labeled chemokines in resting AD derived T cell lines and clones. Percentages of CD4⁺, CCL18-binding T cells are given in upper right quadrant.

FIG. 9: CCL18 binding of AD derived memory T cells. FACS staining for CCR3 on CCL18-binding T cell line NIT

FIG. 10: CCL18 binding of AD derived memory T cells. FACS of CCL18-F- and CCL22-F-binding in the T cell line NIT. All FACS stainings are representative of 5-10 replicates and were consistent among the different T cell lines and clones.

FIG. 11: AD derived memory T cells migration in response to CCL18 in vitro and in vivo. T cells derived from AD skin respond significantly (p<0.05) in a chemotaxis chamber assay to increasing concentrations of CCL18 (filled circles) and CCL22 (empty squares). Highest concentrations are blocked by co-incubation with specific neutralizing mAb. Each point is assessed in quadruplicate and represented as mean ± SE of. 2 out of 15 experiments are shown.

FIG. 12: AD derived memory T cells migration in response to CCL18 in vitro and in vivo. Migration of human memory T cells to human skin grafts in SCID-hu Skin mice. CCL18 (n=8), CCL22 (n=8) or PBS (n=9) are injected into human skin grafts and skin homing of memory T cells assessed by FACS analysis. CCL18 induced significant migration of T cell line NIT and T cell clone 98016T.02 (p<0.05). Two out of 4 independent experiments shown.

In the following examples temperatures are in degree centigrade (° C.) and are uncorrected. The following abbreviations are used:

AD atopic dermatitis

APC antigen presenting cells

BSA bovine serum albumin

CFSE carboxy-fluorescein diacetate succinimidyl

DC dendritic cells

EASI Eczema Area Severity Index

ELISA enzyme linked immunosorbent assay

FACS fluorescence activated cell sort

FACS buffer PBS containing 2% FCS and 0.1% NaN₃

FCS fetal calf serum

FITC fluorescein isothiocyanate

IGA Investigators' Global Assessment

IL interleukin

LC Langerhans cells

mAb monoclonal antibody

PBMC peripheral blood mononuclear cells

PBS phosphate buffered saline

RPMI cell medium developed by the Roosevelt Park Memorial Institute

RT room temperature

SCID severe combined immunodeficiency

SCID-hu Skin mice SCID mice transplanted with human skin

SD standard deviation

SE standard error

Th T helper cells

### EXAMPLES

Methods:

Patients

Blood samples are obtained from 36 patients with AD and 28 non atopic healthy controls. Clinical severity is documented by the EASI and the IGA (see e.g. Hanifin, J. M., et al, Exp. Dermatol. 2001, 10:11-18). Composition of patient population by IGA scores: almost clear: n=3, mild disease: n=0, moderate disease: n=15, severe disease: n=10, very severe disease: n=4. The patients did not receive any systemic or topical treatment with immunosuppressive drugs for at least 4 weeks prior to collection of blood samples and skin biopsies.

Purification and Culture of Cells

PBMC from human blood are prepared by density gradient separation on Ficoll-Paque™ Plus (Amersham Biosciences). The AD derived T cell lines (NIT, SVT, DK2-JOT) and clones (98016T.02, 98016T.24), which express cutaneous lymphocyte antigen (CLA), CCR4 and display a Th2/Th0 cytokine secretion pattern following activation, are generated from lesional punch biopsies, e.g. as described in
Carballido, J. M., et al, J. Immunol. 1997,159:4316-4321,
Biedermann, T., et al,. Eur. J. Immunol. 2002, 32:3171-3180.

T cells are cultured in X-Vivo15 medium (BioWitthaker) supplemented with recombinant human IL-2 (20 ng/ml).

Flow Cytometry

Cells are stained for 30 minutes on ice using FACS buffer. The following mAbs and corresponding isotypes are used: CD3-Pe, CD3-Percp, CD4-Pe, CD4-Percp, CD8-Percp, CD14-FITC, CD19-FITC, CD20-FITC, CD62-Pe, CD56-FITC, HLADR-Cychrome, CCR4-Pe (BD Pharmingen), CD3-APC, CD3-FITC, CD4-APC, CD11a-Pe, CD11c-APC, CD19-APC, (Caltag), CCR3-Pe, CCR5-Pe, CCR7-Pe, CXCR3-Pe, CXCR4, CXCR6-Pe (R&D Systems). CCL22-Alexa647 is provided from Dictagene. CCL18 (Peprotech) is labeled in with FITC or biotin (Molecular Probes) according to manufacturers instructions. Analysis is performed on a FACSCalibur™ (BD) using BD CELL ™Quest Software.

Detection of Intracellular CCL18 in Monocytes and Dendritic Cells After Stimulation with IL-4

PBMC from AD patients (n=8) and controls (n=13) are stimulated (5×10⁶ cells/ml) for 48 hours with or without 10 ng/ml of recombinant human IL-4 and treated with Brefeldin-A (10 μg/ml, Sigma) for the last 3 hours. Monocytes are recognized by CD14 staining, and myeloid DC are identified as the CD11c⁺ fraction of HLA-DR^{+, lin(CD}3,14,19,20,56)⁻ PBMC.

For intracellular staining of CCL18, surface stained cells are fixed with 4% paraformaldehyde (Sigma), permeabilized with 0.5% saponin (Sigma) and stained with goat anti-human CCL18 mAb followed by donkey anti-goat-Pe (DPC Biermann). Goat serum (Sigma) is used as isotype control.

ELISA

Serum CCL18 and CCL22 levels are determined using Duoset ELISA assays.

Chemotaxis Assay

Chemokines or control medium (RPMI 1640 with 0.5% BSA) are added in 30 μl to the lower wells of a 96-well chemotaxis chamber (Neuro Probe). Calcein-AM (1 μM, Molecular Probes) labeled cells are seeded at 3×10⁶ cells/ml in the upper chamber and selected wells of the lower chamber to obtain 100% migration value. The two chamber compartments are separated by a 3 μm pore size polycarbonate filter (Neuro Probe). After 2.5 hours incubation at 37°, filters are removed, the plate is spun down and supernatants discarded. 20 μl of 0.4% Triton-X100 (Sigma) are added to each well. The degree of migration is quantified by fluorescence reading using a Victor II (Wallac) multiwell plate reader.

In Vivo Migration Assay in SCID-hu Skin Mice

SCID (C.B-17/GbmsTac-Prkdc^{scid} Lyst^{bg}) mice (Taconic M&B) are transplanted as described in Carballido, J. M., et al, J. Immunol. Methods 2003, 273:125-135, with human skin pieces obtained from the WHRTB, Petz Aladá r County Hospital Györ, Hungary, according to institutional and governmental ethical guidelines. Chemokine induced skin homing of human T cells is performed using a modification of the method described in Biedermann, T., et al,. Eur. J. Immunol. 2002, 2:3171-3180. AD skin derived T cells are labeled with CFSE (Molecular Probes, 1 μM in PBS for 5 minutes at RT) and 5×10⁷ cells/mouse injected i.v. into SCID mice transplanted with human skin (SCID-hu Skin mice). Immediately thereafter, CCL18 or CCL22 (300 ng in 30 μl PBS, R&D) or PBS alone are injected intradermally into human skin grafts. After 24 hours, skin grafts are explanted, processed into single-cell suspensions, stained and analyzed in the FACS by quantifying the percentages of CD3⁺, CFSE⁺ cells present in the living (propidium iodide negative) cell fraction.

Immunohistology

Acetone fixed 5 μm cryostat sections of normal skin of healthy subjects (n=3) and lesional skin from patients with AD (n=10) or psoriasis (n=3) are stained by goat anti-CCL18 mAb (R&D) or goat serum and blotinylated horse anti-goat IgG (Vector) followed by avidin-conjugated alkaline phosphatase (AP) and naphtol-AS phosphate/Fast Red TR substrate (Sigma). CCL18-binding cells in AD samples are detected using CCL18-biotin followed by avidin-AP and Fast Red. The samples are co-stained with anti-CD3 mAb (BD) followed by anti-mouse-Ig-biotin (Vector), avidin-AP and Fast Blue (Sigma) and counter-stained by hematoxylin eosin (HE, Merck). For immunofluorescence analysis, cryostat sections are stained with goat anti-CCL18 and mouse anti-HLA-DR or anti-CD3 (BD), anti-CD1a (Caltag), anti-CD207 (Baxter) followed by rabbit anti-goat-TxRd and horse anti-mouse-FITC (Vector), respectively. Slides are coversliped with DAPI containing mounting medium (Vector).

Statistical Analysis

Data are presented as means ±SE or boxplots (indicating average, median, 25%-75% percentiles and SD). Statistical significance is determined using unpaired Student's t test. Clinical data are tested for simple correlations by determining Pearson's correlation coefficient and uncorrected probability values. P<0.05 is considered as significant.

### Example 1

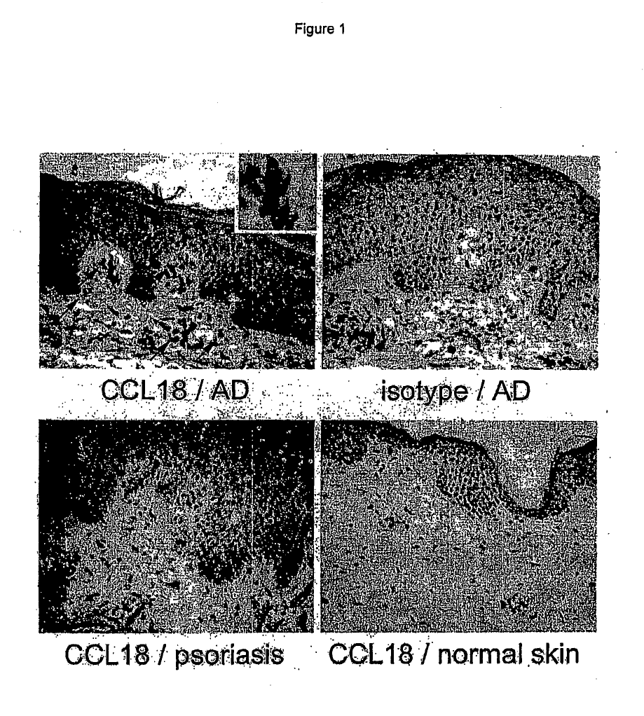

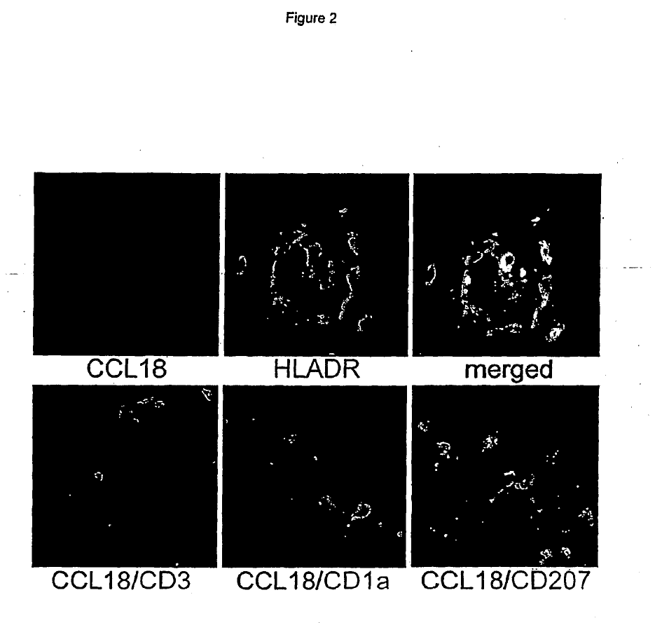

AD is an Inflammatory skin disease associated with cutaneous hyperreactivity to allergens and high IL-4 production. Consequently, AD can provide a suitable environment for CCL18 upregulation. To demonstrate this, an immunohistochemistry evaluation of CCL18 expression in skin samples of AD patients (n=10) in comparison to normal (n=3) and psoriatic (n=3) skin is performed. CCL18 expression is detected in all AD skin samples but is absent in the skin from healthy or psoriatic individuals (see e.g. FIG. 1). In AD skin, CCL18 is strongly expressed in the upper dermal vascular plexus, in close association with the mononuclear cell infiltrates. Immunofluorescence staining demonstrates that CCL18 expression is restricted to HLA-DR⁺APC (see e.g. FIG. 2). Particularly, all CCL18-expressing cells in the epidermis are CD1a⁺. Subsequent staining with CD207 demonstrates that most of the CCL18-expressing cells are LC whereas the remaining population consists of inflammatory dendritic epidermal cells. The production of CCL18 by LC seems to be a result of the Th2 cytokine environment associated with AD, because LC in normal or psoriatic skin does not produce this chemokine. Keratinocytes, fibroblasts and CD3 positive T cells do not express CCL18 (see e.g. FIG. 2).

### Example 2

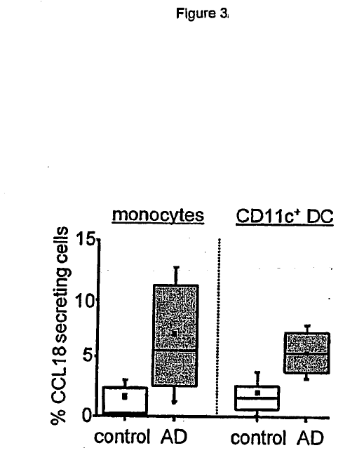

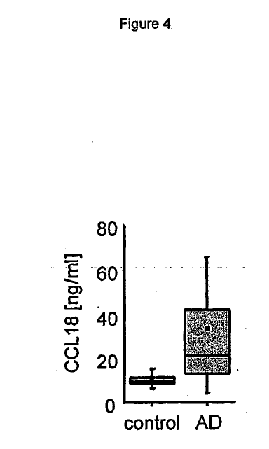

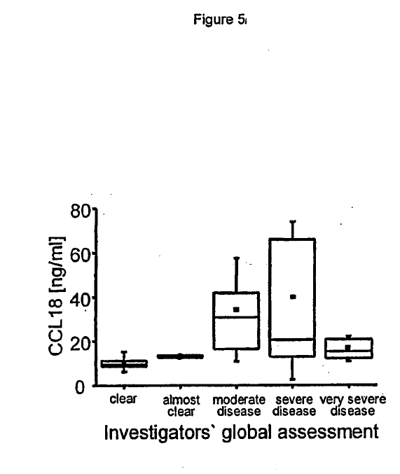

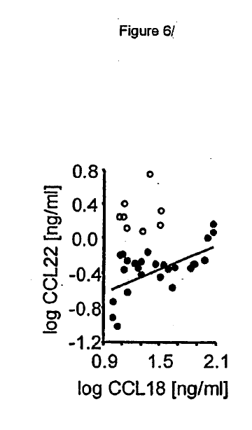

Further investigation shows that CCL18 and CCL18-secreting APC are increased in blood of AD patients. CCL18 expression by APC of AD patients and healthy controls is assessed by intracellular immunofluorescence staining following IL-4 stimulation. We have found that the percentages of CCL18-secreting monocytes (CD14⁺) in blood of AD patients are significantly higher (>5 fold, p<0.003) than those observed in healthy individuals (see e.g. FIG. 3). Similarly, the percentages of CD11c⁺, HLA-DR⁺, in CCL18 producing DC are also Increased (3 fold, p<0.001) in the blood of AD patients (see e.g. FIG. 3). In both groups, CCL18-expressing cells are only detected following stimulation with IL-4. CCL18 production is undetectable in the CD3⁺ T cell and CD19⁺ B cell fractions of unstimulated or IL-4 stimulated PBMC. The higher numbers of CCL18-secreting cells present in the circulation of AD patients correlated with elevated serum levels of CCL18 (see e.g. FIG. 4). Serum values of CCL18 are three fold higher (p<0.0003) In AD patients (mean 34.9 ng/ml±5.3 SE) than in healthy, non allergic, age and sex matched controls (mean 10.7 ng/ml±0.9 SE). The expression levels of CCL18 in moderate and severe AD stages (evaluated by the clinical scores IGA and EASI) are higher than in mild disease manifestations (see e.g. FIG. 5). However, the serum levels of CCL18 decreases in patients with very severe disease scores (see e.g. FIG. 5). Very severe/chronic manifestations of AD do not involve exclusively IL-4 production since many IFN-γ producing Th cells are detected in these skin lesions (see e.g. Grewe, M., et al, Immunol. Today 19:359-361, 1998). It is expected that in severe/chronic situations, when the environment is less rich in IL-4, lower amounts of CCL18 are produced. This can be the reason for the reduction in CCL18 expression observed in the serum of patients with the highest clinical disease scores. In our patient population, we also found a correlation between EASI and serum CCL22 levels (r=0.4, p<0.05, not shown). Furthermore, we found a strong correlation (r=0.6, p<0.003, FIG. 6, filled circles) between CCL18 and CCL22 expression in patients with mild to severe disease. However, patients with highest CCL22 serum levels, who also had the highest disease scores, showed only moderate CCL18 expression (see e.g. FIG. 6, empty circles). Thus, the selective increase of CCL18 expression during the development of AD and its subsequent decline in patients with the highest clinical disease scores suggests an unique pattern of CCL18 regulation.

### Example 3

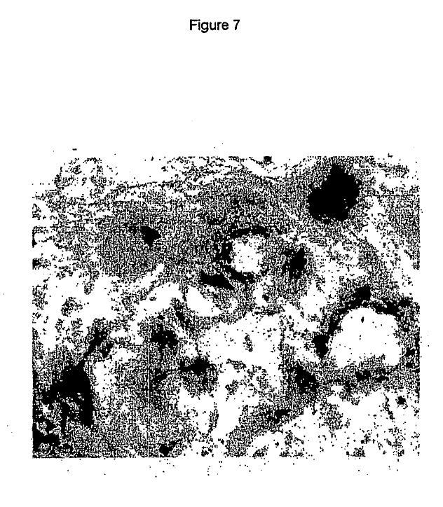

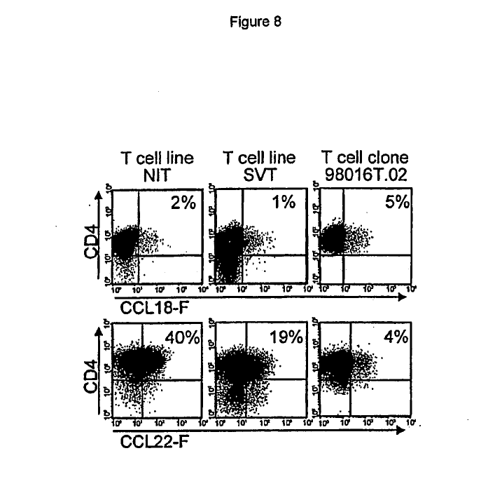

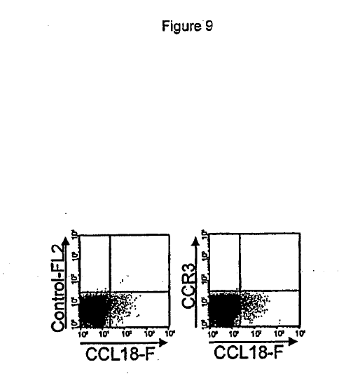

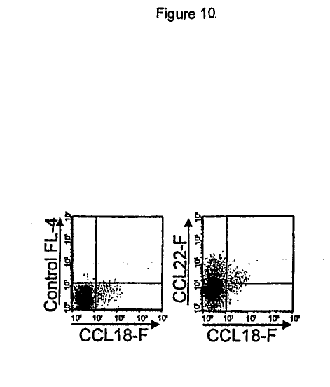

We have also found that CCL18 binds to memory Th cells in AD. The detection of the cell population targeted by CCL18 is hampered by the fact that the CCL18 receptor is not known. To overcome this problem, we generated FITC and blotin labeled CCL18 without altering their biological properties. CCL18 binds to a fraction of CD3⁺ skin infiltrating T cells in biopsies of AD patients (see e.g. FIG. 7). In addition, CCL18 binds to lymphatic vessels, implicating that this chemokine may be involved in lymphocyte trafficking through these channels (see e.g. FIG. 7). CCL18 also binds to up to 5% of memory T cell lines and clones derived from AD skin (see e.g. FIG. 8). Among the T cell lines, CCL18 expression is preferentially observed in the CD4⁺ compartment (see e.g. FIG. 8). The binding is specific since CCL18 also binds to a distinct population of human CD45RA⁺, CCR7^{high}, CD62L^{high}, CD25⁻, CD4⁺ or CD8⁺ T cells in PBMC, which is in agreement with the reported effects of CCL18 on naïve T cells, see e.g. Adema, G. J., et al, Nature 387:713-717 (1997). In addition, CCL18-binding could be competed with unlabeled CCL18 but not with other chemokines including CCL11, which receptor, CCR3, has been described to mediate antagonistic actions of CCL18 (see e.g. Nibbs, R. J., et al, J. Immunol. 2000, 164:1488-1497). Furthermore, using specific mAbs, CCR3 expression on the CCL18-binding T cell population is excluded (see e.g. FIG. 9). Among the different T cell lines and clones, the percentages of CCL22-binding cells are higher than the percentages of CCL18-binding cells (see e.g. FIG. 3B). On the other hand, all CCL18-binding cells also bind CCL22 (see e.g. FIG. 10) suggesting that they may represent a subpopulation of CCR4⁺Th cells.

### Example 4

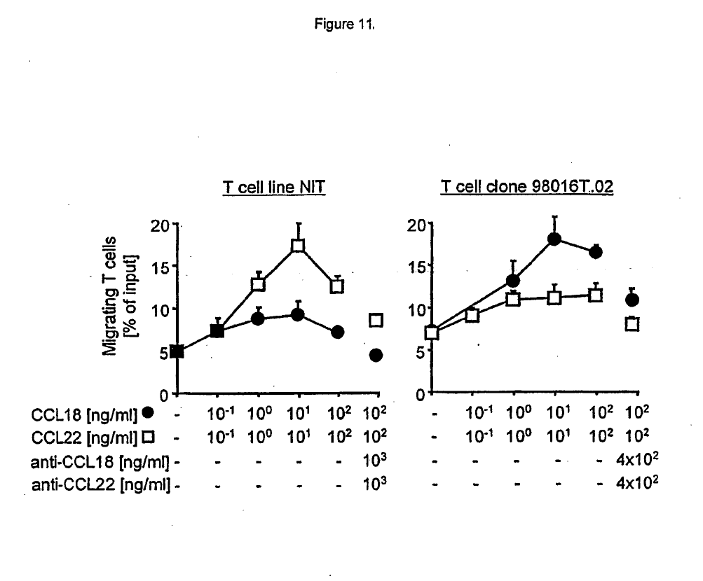

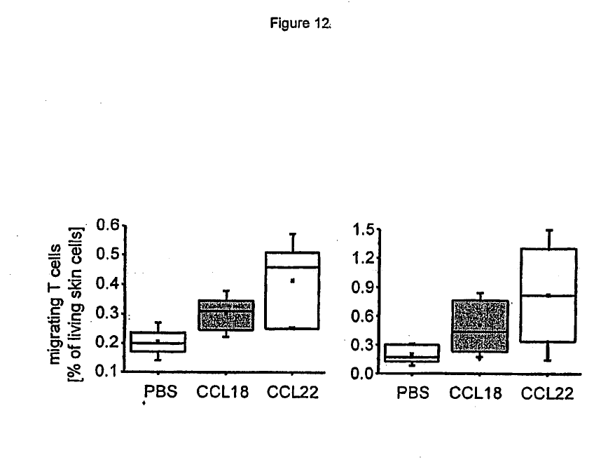

The functional relevance of CCL18-binding to memory Th cells is assessed in functional in vitro and in vivo migration assays. In vitro, all AD-derived T cell lines and clones tested, migrate significantly and in a dose dependent manner in response to CCL18. The extent of CCL18-induced migration is comparable to that induced by CCL22 (see e.g. FIG. 11). Both, CCL18 and CCL22 induced migration, could be blocked using specific neutralizing mAbs, demonstrating the specificity of the responses (see e.g. FIG. 11). The role of CCL18 in vivo is investigated using a modification of the previously described SCID-hu Skin mouse model (Biederman T. et al, Eur. J. Immunol. 2002, 32, 3171-3180). AD derived memory T cell lines and clones migrate significantly to human skin grafts transplanted onto SCID mice in response to CCL18 (see e.g. FIG. 12). CCL18-induced skin homing is comparable in magnitude to the migration mediated by CCL22 (see e.g. FIG. 12).

In conclusion, all these results provide evidence linking CCL18 protein expression with AD and describing a novel capacity of CCL18 to recruit a memory Th cell population into human skin In vivo. The modulation, e.g. enhancement, of CCL18 levels which may be determined in samples from patients, e.g. blood samples, may thus be used as an indication of AD.

1: The chemokine CCL18 for use as a biomarker in a sample of a body fluid of an individual.

2: The chemokine of claim 1 for use as a biomarker in a sample of a body fluid of an individual for a disorder or disease selected from the group consisting of allergic disease and Th2 mediated disorder.

3: The chemokine of claim 1 wherein the disorder or disease is related with atopic dermatitis.

4: Method for screening and/or in vitro diagnosing of a disorder or disease selected from the group consisting of allergic disease and Th2 mediated disorder in an individual, which method comprises
a) providing a sample of a body fluid of an individual,
b) determining the level of CCL18 in said sample as provided in step a),
c) comparing the level of CCL18 as determined in step b) with a reference level from a sample of a body fluid of a healthy control individual, and
d) screening and/or in vitro diagnosing a disorder or disease selected from the group consisting of allergic disease and Th2 mediated disorder in determining whether the level of said CCL18 as determined in step b) is significantly different from said reference level.

5: A method of claim 4 wherein the level of CCL18 is determined in using a CCL18 specific antibody.

6: Method for monitoring the therapeutic efficacy of the treatment of an individual with a substance which is expected to have an effect on reducing or curing a disorder or disease selected from the group consisting of allergic disease and Th2 mediated disorder, which method comprises determining the level of CCL18 in a sample of a body fluid of said individual and comparing it to the level of CCL18 prior to administration of said substance.

7: Kit for screening and/or in vitro diagnosing of a disorder or disease selected from the group consisting of allergic disease and Th2 mediated disorder in a sample of a body fluid of an individual comprising
a) a molecule which recognizes CCL18 protein or a part thereof, optionally in a labeled form,
b) instructions for use,
c) optionally detection means, and
d) optionally a solid phase.
